(19) United States
(12) Patent Application Publication
(10) Pub. No.: US 2008/0213220 A1
(43) Pub. Date: Sep. 4, 2008

(54) Cancer-targeted viral vectors

(76) Inventors: Paul B. Fisher, Richmond, VA (US); Devanand Sarkar, Richmond, VA (US)

Correspondence Address: BAKER BOTTS L.L.P., 30 ROCKEFELLER PLAZA, 44TH FLOOR, NEW YORK, NY, 10112-4498, US

(21) Appl. No.: 11/827,388

(22) Filed: Jul. 11, 2007

Related U.S. Application Data
(63) Continuation-in-part of application No. PCT/US06/00941, filed on Jan. 11, 2006.
Application No. PCT/US06/00941 is a continuation of application No. 11/032,757, filed on Jan. 11, 2005.

(51) Int. Cl. A61K 48/00; C12N 15/861; A61P 35/00

(52) U.S. Cl. 424/93.2; 435/456; 435/320.1

(57) ABSTRACT

The present invention relates to viral vectors that are targeted to cancer cells. The viral vectors of the invention are adenoviruses having a PEG-3 promoter driving the expression of the viral genes E1A and E1B. The PEG-3 promoter exhibits increased activity in malignant cells. Adenoviruses of the invention show increased replication in malignant cells, thereby producing a cytopathic effect. The viral vectors of the invention further comprise additional genes of interest, and/or may have altered capsid proteins that may enhance infection of and/or target infection to cancer cells. Additional cell types derived from diseased states in which the PEG-3 promoter is selectively active are also therapeutic targets of the viral vectors of the instant invention including those generating allergic, autoimmune and inflammatory responses.

### CROSS-REFERENCE TO RELATED APPLICATIONS

This application is a continuation-in-part of International Patent Application No. PCT/US2006/000941, filed Jan. 11, 2006 and published Jul. 20, 2006 as International Publication No. WO 2006/076408, which is a continuation of U.S. application Ser. No. 11/032,757, filed Jan. 11, 2005, each of which is incorporated by reference herein.

### GRANT INFORMATION

The subject matter disclosed herein was developed, at least in part, with the support of National Institutes of Health Grants CA35675, CA97318, CA98712 and/or CA104177 from the U.S. Department of Health and Human Services, so that the United States Government holds certain rights herein.

### 1. INTRODUCTION

The present invention relates to viral vectors that are targeted, by virtue of selective replication and/or selective infection, to cancer cells. In particular, the viral vectors of the invention are adenoviruses having a PEG-3 promoter driving the expression of the viral genes E1A and E1B. Since the PEG-3 promoter is a promoter that exhibits increased activity in malignant cells, the adenoviruses of the invention show increased replication in malignant cells, thereby producing a cytopathic effect. A viral vector of the invention further comprises an additional genes of interest, and/or may have an altered capsid protein that enhances infection of and/or targets infection to cancer cells. Additional cell types derived from diseased states in which the PEG-3 promoter is selectively active are also therapeutic targets of the viral vectors of the instant invention.

### 2. BACKGROUND OF THE INVENTION

### 2.1 Progression Elevated Gene-3

Progression Elevated Gene-3 (PEG-3) was cloned from a tumor progression model based on rat embryo cells E-11 and E11-NMT (Babiss et al. (1985) Science 228, 1099-11101; Fisher et al. (1978) Proc Natl Acad Sci USA 75, 2311-2314; Su et al. (1997) Proc Natl Acad Sci USA 94, 9125-9130). E11 is a mutant adenovirus type 5 (H5ts125)-transformed rat embryo fibroblast cell clone that forms small, slow-growing and compact tumors. E11-NMT is a clone of E11 that has been selected for aggressiveness by passage through a nude mouse and forms rapidly growing, highly aggressive tumors (Babiss et al. (1985) Science 228, 1099-1101). Subtraction hybridization of an E11 cDNA library from an E11-NMT cDNA library identified PEG-3 (Su et al. (1997) Proc Natl Acad Sci USA 94, 9125-9130), which is a C-terminal truncated mutant form of the rat Growth Arrest and DNA Damage Inducible gene-34, (GADD-34) (Hollander et al. (2003) Oncogene 22, 3827-3832).

The promoter region of the PEG-3 gene (PEG-3 promoter) was cloned to investigate the mechanism of induction of PEG-3 expression as a consequence of oncogenic transformation (Su et al. (2000) Oncogene 19:3411-3421; Su et al. (2001) Nucleic Acids Res 29:1661-1671; U.S. Pat. No. 6,472,520 by Fisher). It has been observed that the PEG-3 promoter is ˜8-10 fold more active in CREF cells transformed with either Ha-ras or v-raf than in the parental CREF cells. A minimum region of the promoter that extends from −118 to +194, (where the transcription initiation site is regarded as +1) has been shown to be sufficient for the increased activity associated with transformation and cancer progression (Su et al. (2000) Oncogene 19:3411-3421; Su et al. (2001) Nucleic Acids Res 29:1661-1671; U.S. Pat. No. 6,737,523 by Fisher et al.).

### 2.2 Treatment-Refractory Prostate Cancer

Prostate cancer represents one of the most important health problems in industrialized countries (Sternberg (2002) Crit Rev Oncol Hematol 43:105-21; Di Lorenzo et al. (2006) Int J Immunopathol Pharmacol 19:11-34). It is the most common cancer and the second leading cause of cancer-related deaths in men in the United States. In 2006, the estimated new prostate cancer cases were 234,460 of which 27,350 men were estimated to die predominantly from metastatic prostate cancer. Therapeutic options vary according to the stage of the disease at the time of presentation and diagnosis. Patients with localized disease may be treated with surgery or radiation, whereas the treatment for patients with metastatic disease is purely palliative (Sternberg (2002) Crit Rev Oncol Hematol 43:105-21; Di Lorenzo et al. (2006) Int J Immunopathol Pharmacol 19:11-34). Hormonal treatment with anti-androgens is the standard therapy for stage 1V prostate cancer, but patients ultimately become non-responsive to androgen ablation (Sternberg (2002) Crit Rev Oncol Hematol 43:105-21; Di Lorenzo et al. (2006) Int J Immunopathol Pharmacol 19:11-34). Current therapy options for patients with hormone-refractory prostate cancer include radiotherapy and cytotoxic chemotherapeutic agents, such as mitoxantrone, estramustine and taxanes (Sternberg (2002) Crit Rev Oncol Hematol 43:105-21; Di Lorenzo et al. (2006) Int J Immunopathol Pharmacol 19:11-34; Dyrstad et al. (2006) Curr Pharm Des 12:819-37). Despite a palliative benefit, none of these approaches provide a beneficial impact on the overall survival of patients. Consequently, no consistently effective therapy exists for these patients mandating the development of novel, more efficacious and innovative treatment approaches, especially those targeting metastasis.

Overexpression of anti-apoptotic proteins, Bcl-2 and Bcl-x_{L}, is a frequent occurrence in prostate cancer development and progression (Colombel et al. (1993) Am J Pathol 143:390-400; Krajewska et al. (1996) Am J Pathol 148:1567-76; Krajewski et al. (1994) Cancer Res 54:5501-7). Bcl-2 immunostaining increases with Gleason grade of prostate cancer and Bcl-2 expression is a predictor of poor prognosis in prostate cancer patients (Bauer et al. (1996) J Urol 156:1511-6; Keshgegian et al. (1998) Am J Clin Pathol 110:443-9; Sullivan et al. (1998) Clin Cancer Res 4:1393-403; Bai et al. (1999) Int Oncol 14:785-91). Moreover, Bcl-2 expression is augmented following androgen ablation and correlates with progression of prostate carcinomas from androgen dependence to androgen independence (McDonnell et al. (1992) Cancer Res 52:6940-4; Beham et al. (1998) Int J Mol Med 1:953-9; Kajiwara et al. (1999) Int J Urol 6:520-5). Bcl-x_{L} overexpression associates with resistance to drug-induced apoptosis in prostate cancer cells (Lebedeva et al. (2000) Cancer Res 60:6052-60; Li et al. (2001) Cancer Res 61:1699-706; Liu et al. (1997) Clin Cancer Res 3:2039-46). Forced overexpression of Bcl-2 has been shown to desensitize prostate cancer cells to apoptotic stimuli (Kyprianou et al. (1997) Int J. Cancer. 70:341-8; Tu et al. (1995) Cancer Lett 93:147-55). Accordingly, strategies that overcome apoptosis-resistance afforded by either Bcl-2 or Bcl-x_{L} overexpression would clearly have important therapeutic implications in treating patients with prostate cancer.

### 3. SUMMARY OF THE INVENTION

The present invention relates to modified adenoviral vectors, the replication of which is facilitated in cancer cells by the incorporation of the PEG-3 promoter, which drives the expression of adenoviral genes E1A and E1B, both necessary for viral replication. In addition, a modified adenovirus of the invention further comprises an additional gene of interest, and/or a capsid protein modified to facilitate infection of and/or targeting to cancer cells or other abnormal cells in which the PEG-3 promoter is selectively active.

In one set of non-limiting embodiments, the present invention provides for methods of using the modified adenoviral vectors of the invention to treat forms of cancer which are refractory to conventional therapies, for example apoptosis-resistant prostate cancer.

### 4. BRIEF DESCRIPTION OF THE FIGURES

FIG. 1A-B: Sequence of the rat PEG-3 promoter (SEQ ID NO:1). This region of DNA consists of 2,614 nucleotides. This DNA sequence contains the putative initiation site of transcription of the rat PEG-3 gene. For luciferase assays an about 2,200 nucleotide region of the PEG-3 promoter was cloned into a luciferase reporter vector. Panel A shows nucleotides 1-1500. Panel B shows nucleotides 1501-2614.

FIG. 2: Sequence of the 2.0-kb PEG-3 promoter. (SEQ ID NO:2). The location of PEA3 and AP 1 elements and the TATA boxes are indicated.

FIG. 3: The 477 nucleotide sequence of the PEG-3 Promoter (−282 to +195) (SEQ ID NO:3) used to make the Terminator Virus. The bold underlined base is the transcription start site.

FIG. 4: Schematic representation of steps involved in constructing a conditionally replicative bipartite Terminator adenovirus. pE1.2 and pE3.1 are shuttle vectors in which PEG-3 promoter driving E1A gene (rPEG-Prom-E1A) and CMV promoter driving IFN-γ (CMV-IFN-γ) are ligated, respectively at the multiple cloning site (MCS). The promoter+transgene cassettes are digested out by a suitable restriction enzyme (R.E.), e.g., AlwNI, BstAPI, DraIII or PflMI and ligated into SfiI-digested adenoviral transfer vector pAd.

FIG. 5: Apoptosis induction by an Interferon-γ expressing Terminator Virus in human pancreatic cancer cell lines. The various cell lines were infected with the indicated Ad at an m.o.i. of 100 pfu/cell and 2 days later stained for Annexin V and analyzed by FACS. Early, indicates early apoptotic cells. Late, indicates late apoptotic and necrotic cells.

FIG. 6: Treatment of human tumor xenografts with an Interferon-γ expressing Terminator Virus. A photograph of the tumor-bearing mice injected with different Ads. (A) 1. Control; 2. Ad.vec; 3. Ad.CMV-E1A; 4. Ad.PEG-E1A; 5. Ad.CMV-IFN-γ; 6. Ad.PEG-IFN-γ; 7. Ad.CMV-E1A-IFN-γ; 8. Ad.PEG-E1A-IFN-γ (Terminator Virus). (B) Photograph of the isolated tumors from the sacrificed animals. (C) Graphical representation of the tumor weight of the sacrificed animals at the end of the experiment.

FIG. 7-A-C: Experimental demonstration that tropism modified Triage-type Ads showing increased infectivity compared to unmodified Ad.GFP.LUC in (A) P69 immortalized prostate epithelial cells; (B) DU-145 and (C) PC-3 human prostate cancer cells. Cells were infected with Ad.GFP.LUC (white bars), Ad.RGD.GFP.LUC (light gray bars), Ad.pK7.GFP.LUC (dark gray bars) and Ad.RGD.pK7.GFP.LUC (black bars) at different m.o.i. (left panels) and at 50 m.o.i. (right panels). The percentage of green cells were analyzed by FACS 24 h post-infection (left panels) and 6 and 24 h post-infection (right panels).

FIG. 8A-G. PEG-Prom promotes Ad replication and transgene expression selectively in prostate cancer cells. The indicated cells were either uninfected (control) or infected with the indicated Ad at an m.o.i. of 1000 vp/cell for replication-competent Ad and 5000 vp/cell for replication-incompetent Ad for 48 h. The expressions of E1A, MDA-7/IL-24 and EF1-α proteins were analyzed by Western blot analyses. The cell lines used were: (A) P69; (B) DU-145; (C) DU-145-BCLx_{L}; (D) PC-3; (E) PC-3-BCLx_{L}; (F) LNCaP: (G) LNCaP-BCLx_{L}. The Ad vectors used were: lane 1=control; lane 2=Ad.vec; lane 3=Ad.CMV-E1a; lane 4=Ad.PEG-E1A; lane 5=Ad.CMV-mda-7; lane 6=Ad.PEG-mda-7; lane 7=Ad.CMV-E1A-mda-7; lane 8=Ad.PEG-E1a-mda-7.

FIG. 9A-G. PEG-Prom driven CRCA (Ad.PEG-E1A-mda-7; CTP) selectively kills prostate cancer cells. The indicated cells were either uninfected (control) or infected with the indicated Ad at the indicated m.o.i. (vp/cell). Cell viability was analyzed by standard MTT assay after 2, 4 and 6 days of infection. The data represent mean ±S.D. The cell lines used were: (A) P69; (B) DU-145; (C) DU-145-BCLx_{L}; (D) PC-3; (E) PC-3-BCLx_{L}; (F) LNCaP: (G) LNCaP-BCLx_{L}. The Ad vectors and m.o.i. used were: lane 1=control; lane 2=Ad.vec-5000; lane 3=Ad.CMV-E1A-10; lane 4=Ad.CMV-E1A-100; lane 5=Ad.CMV-E1A-1000; lane 6=Ad.PEG-E1A-10; lane 7=Ad.PEG-E1A-100; lane 8=Ad.PEG-E1A-1000; lane 9=Ad.CMV-mda-7-1000; lane 10=Ad.CMV-mda-7-2000; lane 11=Ad.CMV-mda-7-5000; lane 12=Ad.PEG-mda-7-1000; lane 13=Ad.PEG-mda-7-2000; lane 14=Ad.PEG-mda-7-5000; lane 15=Ad.CMV-E1A-mda-7-10; lane 16=Ad.CMV-E1A-mda-7-100; lane 17=Ad.CMV-E1A-mda-7-1000; lane 18=Ad.PEG-E1a-mda-7-10; lane 19=Ad.PEG-E1a-mda-7-100; lane 20=Ad.PEG-E1a-mda-7-1000.

FIG. 10A-G. PEG-Prom driven CRCA (Ad.PEG-E1A-mda-7; CTV) selectively induces apoptosis in prostate cancer cells. The indicated cells were treated as in FIG. 8A-G. Annexin V staining was analyzed by flow cytometry 48 h after infection. The cells used were: (A) P69; (B) DU-145; (C) DU-145-BCLx_{L}; (D) PC-3; (E) PC-3-BCLx_{L}; (F) LNCaP: (G) LNCaP-BCLx_{L}. The Ad vectors used were: lane 1=control; lane 2=Ad.vec; lane 3=Ad.CMV-E1a; lane 4=Ad.PEG-E1A; lane 5=Ad.CMV-mda-7; lane 6=Ad.PEG-mda-7; lane 7=Ad.CMV-E1A-mda-7; lane 8=Ad.PEG-E1a-mda-7.

FIG. 11A-D. PEG-Prom driven CRCA (Ad.PEG-E1A-mda-7; CTV) eradicates primary and distant tumors. Subcutaneous tumor xenografts from DU-145-Bcl-x_{L} cells were established in athymic nude mice in both left and right flanks and only tumors on the left side were injected with PBS (control) or with the indicated Ad for 3 weeks (total of seven injections). (A) Measurement of tumor volume in left flank. (B) Measurement of tumor volume in right flank. For (A) and (B), the data represent mean ±S.D. with a minimum of 5 mice in each group. (C). Photograph of the animals of each representative group. The white arrow indicates the tumor. (D). Left panel: photograph of the tumor at the end of the study. Right panel: measurement of tumor weight at the end of the study. The data represent mean ±S.D. with at least 5 mice in each group.

FIG. 12A-B. PEG-Prom driven CRCA (Ad.PEG-E1A-mda-7; CTV) replicates at distant sites and generates MDA-7/IL-24 protein. Tumors were harvested after 3 injections of Act. Formalin-fixed paraffin embedded sections were immunostained for (A) E1A and (B) MDA-7/IL-24.

FIG. 13A-B. PEG-Prom driven CRCA (Ad.PEG-E1A-mda-7; CTP) inhibits angiogenesis. Tumors were harvested after 3 injections of Ad. Formalin-fixed paraffin embedded sections were immunostained for CD31 (A) Confocal images of the tumors stained for CD31. (B) Quantification of microvessel density in the tumors. The data represent mean ±S.D.

### 5. DETAILED DESCRIPTION OF THE INVENTION

The present invention relates to modified recombinant adenovirus vectors comprising (i) a PEG-3 promoter operably linked to the E1A gene and (ii) an additional gene of interest, operably linked to a promoter which is constitutively active or inducibly active in the intended target cell.

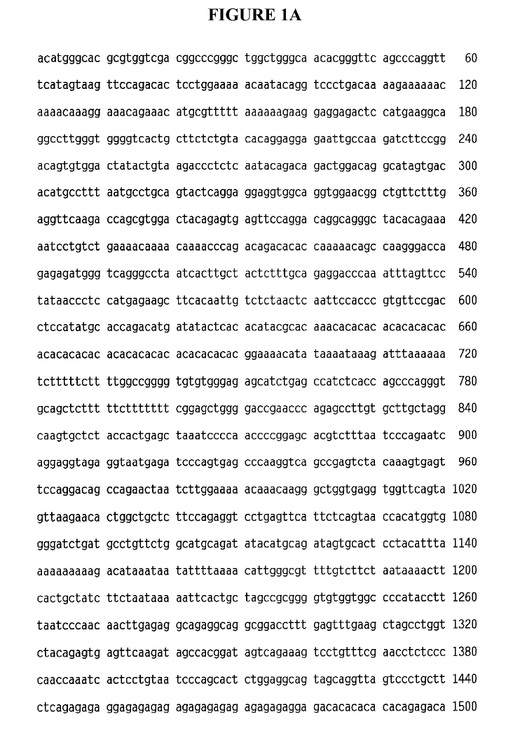

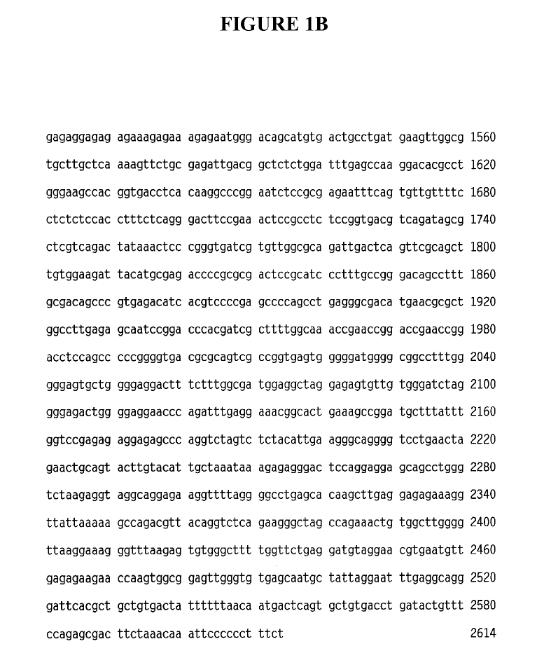

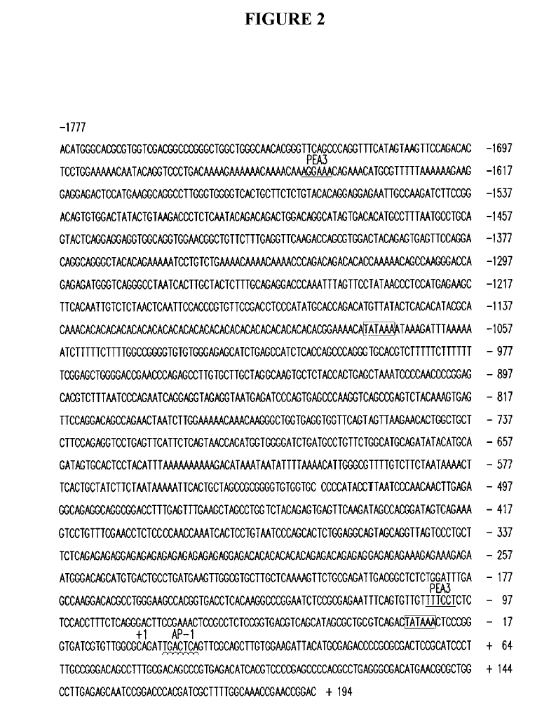

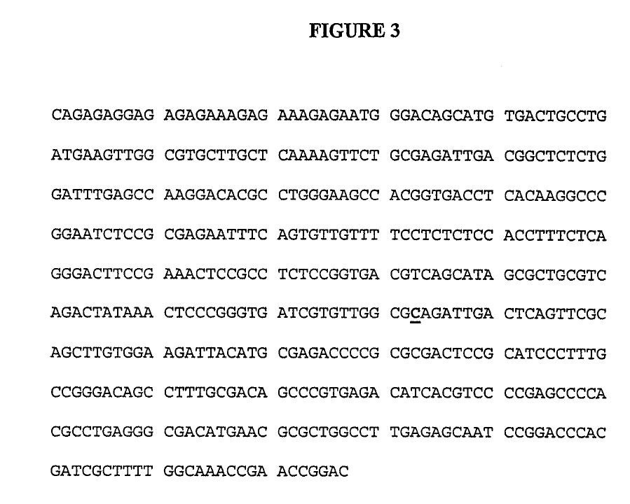

PEG-3 promoters which may be used according to the invention are disclosed in U.S. Pat. Nos. 6,737,523 and 6,472,520. A PEG-3 promoter, according to the invention, may be a rat PEG-3 promoter having SEQ ID NO:1, as depicted in FIGS. 1A and 1B, or may be an improved rat PEG-3 promoter that comprises the core active regions. An improved rat PEG-3 promoter preferably comprises (i) a PEA3 protein binding sequence consisting of the nucleotide sequence beginning with the thymidine (T) at position −105 and ending with the thymidine (T) at position −100 of FIG. 2 (nucleotides 1672-1677 of SEQ ID NO:2), (ii) a TATA sequence consisting of the nucleotide sequence beginning with the thymidine (T) at position −29 and ending with the adenosine (A) at position −24 of FIG. 2 (nucleotides 1748-1753 of SEQ ID NO:2), or (iii) an API protein binding sequence consisting of the nucleotide sequence beginning with the thymidine (T) at position +5 and ending with the adenosine (A) at position +11 of the nucleotide sequence shown in FIG. 2 (nucleotides 1781-1787 of SEQ ID NO:2). In another embodiment, the nucleic acid comprises at least two of the nucleotide sequences (i) to (iii) listed above. In a specific non-limiting embodiment, an improved rat PEG-3 promoter is a nucleic acid molecule having SEQ ID NO:3 (FIG. 3), PEG-3 promoter coordinates −282 to +195.

A PEG-3 promoter of the invention may also be a nucleic acid molecule that is at least about 85 percent, at least about 90 percent, or at least about 95 percent homologous to SEQ ID NO:1, SEQ ID NO:2 or SEQ ID NO:3 (“about” herein means ±20 percent of the recited value), and/or that hybridizes to a nucleic acid molecule having SEQ ID NO:1, SEQ ID NO:2 or SEQ ID NO:3 or its complementary strand under stringent conditions for detecting hybridization of nucleic acid molecules as set forth in “Current Protocols in Molecular Biology”, Vol. I, Ausubel et al., eds. John Wiley: New York N.Y., pp. 2.10.1-2.10.16, first published in 1989 but with annual updating, wherein maximum hybridization specificity for DNA samples immobilized on nitrocellulose filters may be achieved through hybridization to filter-bound DNA or RNA in 0.5 M NaHPO4, 7% sodium dodecyl sulfate (SDS), 1 mM EDTA at 65° C., and washing twice or more in 0.1×SSC (15-30 mM NaCl, 1.5-3 mM sodium citrate, pH 7.0)/0.1% SDS at 68° C. For DNA or RNA samples immobilized on nylon filters, a stringent hybridization washing solution may alternatively be comprised of 40 mM NaPO4, pH 7.2, 1-2% SDS and 1 mM EDTA, for which a washing temperature of at least 65-68° C. is recommended.

To be “operably linked” to the E1A and E1B genes of an adenovirus, the PEG-3 promoter is positioned upstream of the E1A coding region. In non-limiting embodiments, the construction of such an adenovirus may be achieved through recombination between a “rescue” plasmid containing an almost complete copy of the viral genome and a “shuttle” plasmid containing a foreign gene or modified viral gene flanked on both sides by regions of the Ad genome wherein the heterologous gene is to be inserted, whereby upon co-transfection and recombination between rescue and shuttle plasmids, a fully functional recombinant viral genome expressing heterologous elements is generated.

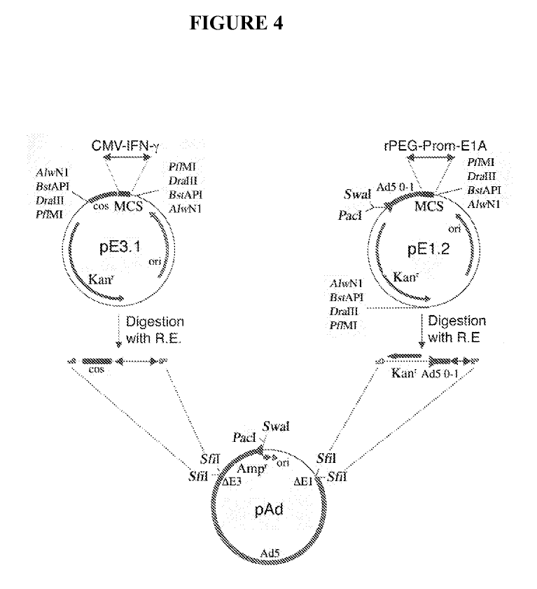

In a specific non-limiting embodiment, constructing the conditionally replicative recombinant adenovirus based on the activity of the PEG-3 promoter comprises the following steps. The PEG-3 promoter is inserted into the multiple cloning site (MCS) of shuttle plasmid pE1.2 (FIG. 4) or an adenoviral shuttle plasmid vector with similar properties. Insertion of the PEG-3 promoter in the MCS results in a gene configuration so as to drive expression of the genes encoded by the E1A region. The PEG-3 promoter driven E1A transcription unit in the pE1.2 or similar shuttle vector is inserted into a rescue vector containing complementary regions of the adenovirus genome e.g. pAd (FIG. 4) or similar adenoviral rescue vector. This step may be accomplished by utilizing compatible flanking restriction enzyme sites e.g. SfiI in pAd. In this non-limiting embodiment, pAd or other related adenoviral rescue vectors may be deleted in the E1A region. Cloning of fragments is achieved by standard DNA ligation or by other means known to those skilled in the art e.g., by Polymerase Chain Reaction (PCR), in vitro or in vivo recombination. By cloning the PEG-3 promoter E1A fragment from pE1.2 into pAd or a related vector, a reconstituted E1A and E1B region controlled by the PEG-3 promoter is generated.

The invention provides for a modified adenovirus having a PEG-3 promoter operably linked to the E1A (and E1B) gene(s) which further comprises an additional active transcriptional unit expressing a heterologous (non-adenovirual) gene of interest. The gene of interest may encode a secreted product or a non-secreted product. Such modified viruses are referred to herein as “Terminator Viruses”. Preferably, said gene of interest may be comprised in the E3 gene of adenovirus. Insertion of an active transcriptional unit comprising a promoter driving a gene of interest into the E3 region may be accomplished, for example, by the following steps. The gene of interest may be inserted into a shuttle vector such as pE3.1 (FIG. 4) or another vector with similar properties, which enables insertion into the E3 region of the adenoviral genome. The transcription unit with the gene of interest may then be excised from the shuttle vector using appropriate compatible restriction enzyme sites (e.g. SfiI). The excised transcription unit expressing the gene of interest may then be cloned into the E3 region of the adenoviral genome utilizing an adenoviral rescue vector exemplified but not limited to pAd (FIG. 4). Selective insertion into the E3 region is achieved via compatible restriction digestion and ligation of pAd vector to the insert fragment or by other means known to one skilled in the art.

A gene of interest may be, for example and not by way of limitation, a gene that augments immunity (in a subject to whom the virus is administered), such as IFN-α, IFN-β, IFN-γ, IL-2, IL-4, IL-12 etc., a gene involved in innate immune system activation such as mda-5 (Kang et al., 2002 Proc Natl Acad Sci USA. 99(2):637-42), RIG-I (Heim, 2005, J Hepatol. 42(3):431-3) etc., a gene that has an anti-cancer effect, including genes with anti-proliferative activity, anti-metastatic activity, anti-angiogenic activity, or pro-apoptotic activity, such as mda-7/IL-24 (Sarkar et al. (2002) Biotechniques Suppl: 30-39; Fisher et al. (2003) Cancer Biol Ther 2:S23-37), TNF-α (Anderson et al. Curr Opin Pharmacol (2004) 4(4):314-320), IFN-β (Yoshida et al, (2004) Cancer Sci 95(11):858-865), p53 (Haupt et al. Cell Cycle (2004) 3(7):912-916), BAX (Chan et al. Clin Exp Pharmacol Physiol (2004) 31(3):119-128), PTEN (Sansal et al. J Clin Oncol (2004) 22(14):2954-63), soluble fibroblast growth factor receptor (sFGFR) (Gowardhan et al. (2004) Prostate 61(1):50-59), RNAi or antisense-ras (Liu et al. Cancer Gene Ther (2004) 11(11):748-756.), RNAi or antisense VEGF (Qui et al. Hepatobiliary Pancreat Dis Int (2004) 3(4):552-557), antisense or RNAi mda-9/syntenin (Sarkar et al. Pharmacol Ther (2004) 104(2):101-115) etc., a gene that renders an infected cell detectable, such as green fluorescent protein (or another naturally occurring fluorescent protein or engineered variant thereof), β-glucuronidase, β-galactosidase, luciferase, and dihydrofolate reductase, or a gene which enhances radiotherapy including but not limited to p53 (Haupt et al. Cell Cycle (2004) 3(7):912-916), GADD34 (Leibermann et al. Leukemia (2002) 16(4):527-41), the sodium iodide symporter (for thyroid cancer) (Mitrofanova et al. Clin Cancer Res (2004) 10(20):6969-6976), etc.

In further non-limiting embodiments a modified adenovirus having a PEG-3 promoter operably linked to the E1A and E1B genes and comprising an additional active transcriptional unit expressing a heterologous gene of interest may be utilized to deliver a therapeutic amount of an anti-inflammatory, anti-allergic or antiviral gene product either systemically or at a specific target site in a human subject or non-human animal. Non-limiting examples of such genes include IFN-α or IFN-β (Markowitz, Expert Opin Emerg Drugs (2004) 9(2):363-374) to treat an inflammatory condition or for anti-viral therapy (Suzuki et al. J Gastroenterol (2004) 39(10):969-974; Malaguamera et al BioDrugs. (2004) 18(6):407-413), Interferon Regulatory Factor-1 (IRF-1) for inflammation (Siegmund et al. Eur J Immunol (2004) 34(9): 2356-2364), mda-5 (Andrejeva et al., 2004 Proc Natl Acad Sci USA. 101(49):17264-9; Yoneyama et al; 2005, J Immunol. 175(5):2851-8) and RIG-I (Meylan et al., 2005, Nature. 437(7062): 1167-72) for antiviral activity or stimulation of the innate immune system etc. In various non-limiting embodiments, a modified adenoviral vector may comprise, as a gene of interest, a gene having a product that enhances, in a subject having a cancer, the immune response of the subject to the cancer. Suitable genes of interest include, but are not limited to, genes encoding tumor-associated antigens recognized by the immune system, such as gp100, PSA, EGFR, CEA, HER-2/neu, CO17-1a, MUC-1, gp72/CD55, gastrin, β-HCG, α-fetoprotein, heat shock protein (gp96), etc. (Mocellin et al. (2004) Gastroenterology 127:1821-1837). Since inadequate or inhibitory T-cell costimulatory pathway signaling has been shown to restrict productive immune responses against cancer cells, genes of interest encoding costimulatory ligands such as B7-H3 (Luo et al. (2004) J Immunol 173(9):5445-5450), GM-CSF/IL-2 fusion protein (Stagg et al. (2004) Cancer Res 64(24): 8795-8799) etc. may be comprised in the modified adenoviruses of the invention.

The gene of interest, located in (inserted into) the E3 or other suitable region of the adenoviral genome, is operably linked to a promoter element which is constitutively or inducibly active in the intended target cell (e.g., a cancer cell in a tumor to be treated). Suitable promoters include, but are not limited to, the cytomegalovirus immediate early promoter, the Rous sarcoma virus long terminal repeat promoter, the human elongation factor-1α promoter, the human ubiquitin c promoter, etc. (Colosimo et al. Biotechniques (2000) 29(2):314-318, 320-322, 324) and the PEG-3 promoter (U.S. Pat. Nos. 6,472,520 and 6,737,523 and as defined herein, including SEQ ID NO: 1, 2 or 3, or molecules that are at least about 85 percent, at least about 90 percent, or at least about 95 percent homologous thereto; Su et al. (2000) Oncogene 19:3411-3421; Su et al. (2001) Nucleic Acids Res 29:1661-1671; provided the gene configuration having a direct repeat of two identical PEG-3 promoter DNA sequences separated by an intervening DNA does not undergo intramolecular recombination). It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. (Romano, Drug News Perspect (2004) 17(2):85-90). In specific non-limiting embodiments of the invention, the promoter may be selectively active in cancer cells, such as the prostate specific antigen gene promoter (O'Keefe et al. (2000) Prostate 45:149-157), the kallikrein 2 gene promoter (Xie et al. (2001) Human Gene Ther 12:549-561), the human alpha-fetoprotein gene promoter (Ido et al. (1995) Cancer Res 55:3105-3109), the c-erbB-2 gene promoter (Takakuwa et al. (1997) Jpn. J. Cancer Res. 88:166-175), the human carcinoembryonic antigen gene promoter (Lan et al. (1996) Gastroenterol. 111: 1241-1251), the gastrin-releasing peptide gene promoter (Inase et al. (2000) Int. J. Cancer 85:716-719). the human telomerase reverse transcriptase gene promoter (Pan and Koenman, 1999, Med Hypotheses 53:130-135), the hexokinase II gene promoter (Katabi et al. (1999) Human Gene Ther 10:155-164), the L-plastin gene promoter (Peng et al. (2001) Cancer Res 61:4405-4413), the neuron-specific enolase gene promoter (Tanaka et al. (2001) Anticancer Res 21:291-294), the midkine gene promoter (Adachi et al. (2000) Cancer Res 60:4305-4310), the human mucin gene MUC1 promoter (Stackhouse et al. (1999) Cancer Gene Ther 6:209-219), and the human mucin gene MUC4 promoter (Genbank Accession No. AF241535), which is particularly active in pancreatic cancer cells (Perrais et al. (2000) J Biol Chem 276(33):30923-30933). In certain non-limiting embodiments of the invention, the promoter operably linked to the gene of interest is not a PEG-3 promoter as defined herein.

In another set of embodiments, a modified adenovirus having a PEG-3 promoter operably linked to the E1A and E1B genes (and optionally an inserted gene of interest) may further comprise a virion fiber or hexon capsid protein modification to facilitate infection of target cells and/or enhance targeting of an adenovirus vector to specific cell types. Such viruses are referred to herein as “Triage Viruses”. Such capsid-modified adenoviruses are generically referred to in the literature as “infectivity enhanced” adenoviruses (Krasnykh et al. Cancer Res (2000) 60(24):6784-6787). Such modifications include but are not restricted to incorporation of targeting ligands within the capsid proteins. The instant invention in a specific embodiment comprises an infectivity enhanced conditionally replicating adenovirus constructed to embody the combined properties of enhanced infectivity and conditional replication dependent on cancer specific expression of the PEG-3 promoter.

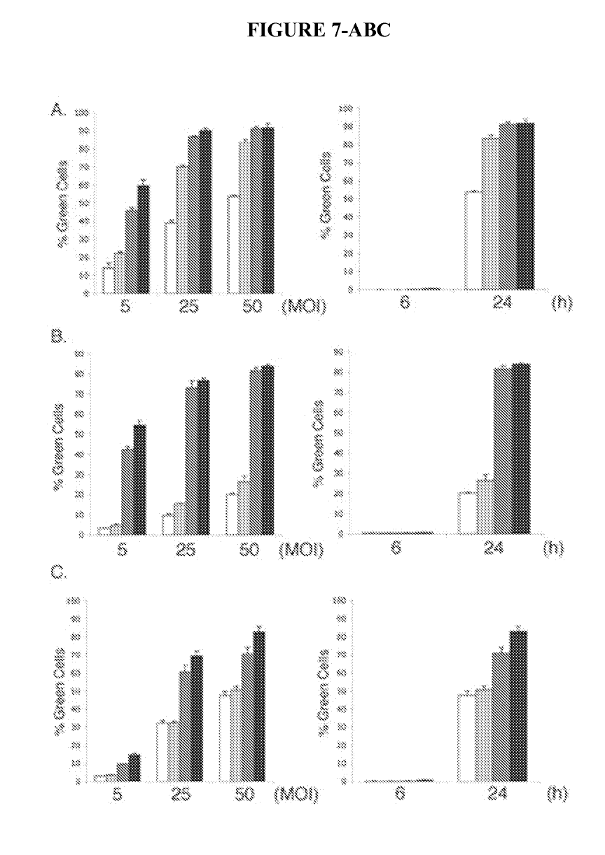

In non-limiting embodiments one or more heterologous targeting ligands may be incorporated within the fiber. Based on the three dimensional model of the fiber knob, targeting ligand may be inserted into the HI loop of the fiber (Ruigork et al. (1990) Mol Biol 215:589-596). This loop is flexible, exposed outside the knob, is not involved in fiber trimerization, and its variable length is different among Ad serotypes suggesting that insertions or substitutions do not substantially affect fiber stability (Krasnyk et al. (1996) J Virol 70:6839-6846; Douglas et al. (1996) Nature Biotech 14:1574-1578). In a specific non-limiting embodiment, two types of ligands may be introduced into the HI loop of the fiber: (i) the sequence coding for an RGD peptide, CDCRGRDCFC (SEQ ID NO:5), known to target tumors by binding with high affinity to several types of integrins thus facilitating binding via fiber-RGD/integrin interaction independent of the adenoviral CAR receptor (Krasnykh et al. Cancer Res (2000) 60(24):6784-6787); and (ii) the sequence encoding a poly-lysine (pK7)-peptide (GSGSGSGSGSKKKKKKK) (SEQ ID NO:4) incorporated at the C terminal of the fiber protein) permitting attachment and entry through heparin sulfate-containing receptors which also facilitate CAR-independent infection (Krasnykh et al. Cancer Res (2000) 60(24):6784-6787). Results shown in FIGS. 7-A-C demonstrate that infectivity of adenoviruses with modified fiber structure as described supra provides higher infectivity in prostate cancer cells.

In further embodiments, the conditionally replicating adenoviral vector may be tropism-modified by altering the nature and properties of the hexon protein (Krasnyk et al. (1996) J Virol 70:6839-6846). The hexon protein is in greater than twenty-fold abundance than the fiber protein. The hexon protein may be modified to contain a small peptide ligand with high specificity for a cellular target. When expressed as a heterologous component of a hexon protein a small peptide ligand is presented on the surface of an adenovirus with high relative abundance. Peptide ligands when presented in this manner overcome potential lack of high affinity through increased avidity. Modification of hexon protein may be accomplished by genetic incorporation of DNA sequences coding for ligands into the hyper-variable regions of the hexon gene utilizing a suitable shuttle vector. In additional non-limiting embodiments, the fiber knob may be altered by genetic incorporation of alternate knob domains (Henry et al (1994) J Virol 68(6):5239-5246; Krasnyk et al. (1996) J Virol 70: 6839-6846).

The present invention further provides a method for producing a cytopathic effect in a cell comprising infecting the cell with a modified adenovirus according to the invention. Types of cytopathic effects include a decrease in cell proliferation, a decrease in cell metabolism, and/or cell death. The cell may be a cancer cell of for example, a nasopharyngeal tumor, a thyroid tumor, a central nervous system tumor (e.g., a neuroblastoma, astrocytoma, or glioblastoma multiforme), melanoma, a vascular tumor, a blood vessel tumor (e.g., a hemangioma, a hemangiosarcoma), an epithelial tumor, a non-epithelial tumor, a blood tumor, a leukemia, a lymphoma, a cervical cancer, a breast cancer, a lung cancer, a prostate cancer, a colon cancer, a hepatic carcinoma, a urogenital cancer, an ovarian cancer, a testicular carcinoma, an osteosarcoma, a chondrosarcoma, a gastric cancer, or a pancreatic cancer. The cell may be a cancer cell in a human or a non-human animal subject. To achieve infection, the amount of modified virus administered may be preferably, but not by way of limitation, between about 1×10¹⁰ to 1×10¹³ pfu or at a multiplicity of infection (m.o.i.) of between about 10 and 5000 virus particles, between about 10 and 1000 virus particles, or between about 100 and 1000 virus particles, per estimated cell (where the tumor volume can be estimated, the number of cells in the tumor may be estimated (e.g., a spherical tumor having a diameter of 1 cm may be estimated to contain 10⁹ cells; see James et al. (1999) JNCI 91:523-528)), and the effective amount may be administered in a series of inoculations, for example, between 1 and 15 inoculations, or between about 3 and 12 inoculations, or between about 3 and 7 inoculations, each containing between about 1×10¹⁰ to 1×10¹² pfu or at a multiplicity of infection (m.o.i.) of between about 10 and 5000 virus particles, between about 10 and 1000 virus particles, or between about 100 and 1000 virus particles, per estimated cell. Where the modified adenovirus is administered to a subject, the mode of administration (inoculation) may be, but is not limited to, intra-tumor instillation, intravenous, intra-arterial, intrathecal, intramuscular, intradermal, subcutaneous, mucosal via pulmonary or other route, direct nasal installation, etc.

In one set of non-limiting embodiments, the present invention provides for methods of using the modified adenoviral vectors of the invention to treat forms of cancer which are refractory to conventional therapies (“refractory cancers”), or to inhibit the proliferation of a cell of a refractory cancer, or to inhibit tumor growth and/or metastasis of a refractory cancer. In one non-limiting set of embodiments, a cancer which has not shown adequate clinical response to a treatment agent, or combination thereof, which is not a modified adenoviral vector of the invention, is considered such a refractory cancer. In another non-limiting set of embodiments, a cancer which overexpresses as antiapoptotic protein, such as but not limited to Bcl-2 or Bcl-x_{L}, is such a refractory cancer. In a specific, non-limiting example, a refractory cancer is apoptosis-resistant and/or treatment resistant prostate cancer. In other specific, non-limiting examples, a breast cancer which overexpresses Bcl-2, a small-cell lung cancer which overexpresses Bcl-2, a non-small cell lung cancer which overexpresses Bcl-2, and a liver cancer which overexpresses Bcl-2, are each considered to be refractory cancers.

Accordingly the present invention provides for a method of treating a subject suffering from a cancer, where the cancer is selected from the group consisting of breast cancer, lung cancer, prostate cancer, colon cancer, rectal cancer, hepatic carcinoma, urogenital cancer, ovarian cancer, testicular carcinoma, osteosarcoma, chondrosarcoma, gastric cancer, pancreatic cancer, nasopharyngeal cancer, thyroid cancer, neuroblastoma, astrocytoma, glioblastoma multiforme, melanoma, hemangiosarcoma, an epithelial cancer, a non-epithelial cancer such as squamous cell carcinoma, leukemia, lymphoma, and cervical cancer, comprising administering, the subject, an effective amount of a modified adenovirus according to the invention. In non-limiting embodiments of the invention, an effective amount of modified adenovirus may be between about 1×10¹⁰ to 1×10¹² pfu or a multiplicity of infection (m.o.i.) of between about 10 and 5000 virus particles, between about 10 and 1000 virus particles, or between about 100 and 1000 virus particles, per estimated cell, and the effective amount may be administered in a series of inoculations, for example, between 1 and 15 inoculations, or between about 3 and 12 inoculations, or between about 3 and 7 inoculations, each containing between about 1×10¹⁰ to 1×10¹² pfu or at a multiplicity of infection (m.o.i.) of between about 10 and 5000 virus particles, between about 10 and 1000 virus particles, or between about 100 and 1000 virus particles, per estimated cell.

“Treating” a subject suffering from a cancer means one or more of the following: decreasing tumor volume; decreasing rate of tumor growth; increasing survival; decreasing tumor grade; inhibiting metastasis (meaning inhibiting dissemination and/or growth/proliferation of metastatic cells), increasing time of survival, and/or improving quality of life (e.g., decreasing pain, increasing ability to perform activities).

The present invention in further non-limiting embodiments provides for a method of treatment of various types of cancer cells described supra involving combined treatment of a Terminator or Triage Virus with radio- or chemotherapeutic agents. PEG-3 promoter activity is enhanced by DNA damaging agents and ionizing radiation (Su et al. (1999) Proc Natl Acad Sci USA 96(26):15115-151120; Su et al. (2002) J Cell Physiol 192(1):34-44). Therefore enhanced viral replication leading to enhanced cytolysis of tumor cells may be achieved. Combination therapy includes but is not limited to simultaneous or serial treatment with a Terminator or Triage Virus embodied in instant invention and standard radiotherapy or chemotherapy regimes. Chemotherapy may include but is not limited to treatment with appropriate doses of chemotherapy agents such as Cisplatin, Adriamycin, Doxorubicin, Paclitaxel or other Taxol derivatives, etc. In an additional embodiment, specific targeting to an organ, tumor or tissue type or enhanced infectivity is obtained by utilizing an appropriate Triage Virus.

In further non-limiting embodiments, a combination of two or more Terminator or Triage Viruses may be used for a method of treatment of a cancer or other disease state. In this embodiment two or more Terminator or Triage Viruses expressing distinct genes of interest may be used in combination (administered concurrently or sequentially) for treatment in a human or non-human animal subject. Non-limiting examples of such combinations include treatment of a subject with two Terminator viruses, one expressing a gene of interest encoding IFN-α, IFN-β, IFN-γ, IL-2, IL-4, IL-12, RIG-I, mda-5 etc. and the other expressing a gene of interest encoding a tumor specific antigen or an immune accessory molecule such as Carcino-Embryonal Antigen (CEA), the B7.1 gene, lymphocyte homing receptor or HLA antigen gene.

In further non-limiting embodiments, Terminator or Triage Viruses expressing appropriate genes of interest may be utilized to restore or boost the responsiveness of a subject to a specific form of conventional radio-, chemo- or immunotherapy. Non-limiting examples of such viruses contain a gene of interest which encodes the EGFR (Epidermal Growth Factor Receptor) or related variants such as the Her-2/neu receptor thereby enhancing a subject's responsiveness to therapies such as Herceptin in breast cancer patients or other anti-EGRF therapies such as Gefitinib (Iressa, ZD1839) an EGFR specific tyrosine kinase inhibitor or the tyrosine kinase inhibitor NVP-AEE788 (AEE788) which blocks both the EGF and VEGF signaling pathways. Viruses containing a gene if interest encoding the androgen receptor (AR) may be used to enhance or restore responsiveness to anti-androgen therapy in androgen refractive forms of prostate cancer. In further embodiments, Triage Viruses that target expression to specific tissues such as breast or prostate and in addition, restore responsive therapeutic targets such as EGFR or AR may be utilized to localize and enhance the efficiency of a particular form of radio-, chemo- or immunotherapy.

In one specific, non-limiting embodiment, the present invention provides for an adenovirus comprising a PEG-3 promoter operably linked to the E1A gene, further comprising mda-7 inserted, operably linked to a promoter, into the E3 region of the adenovirus.

For clarity but not by way of limitation, definitions of terms utilized to describe the various activities of Terminator and Triage Viruses described above are as follows:

(1) Anticancer activity may be defined as the destruction and/or inhibition of proliferation and/or promotion of differentiation of cancer cells. Cancer cells are malignantly transformed cells known to those skilled in the art as cells with known and unknown abnormalities in growth regulatory genes and pathways. Such cells possess the capacity to grown in an unregulated manner and may give rise to tumor formation in naturally occurring disease conditions or under experimental conditions. A tumor is defined as a homogenous or heterogeneous mass of cancer cells. Anticancer activity includes destruction and/or inhibition of proliferation and/or promotion of differentiation of cancer cells grown in vitro or cancer cells in a subject including a human or non-human animal. Destruction and/or inhibition of proliferation and/or promotion of differentiation of cancer cells may involve mechanisms known to those skilled in the art including but not limited to various pathways of differentiation, apoptosis (programmed cell death) or necrosis. Anticancer activity may further involve destruction and/or inhibition of proliferation and/or promotion of differentiation of disseminated cancer cells also known as metastatic cancer cells that have the capacity to move away form the site of an initial tumor and may be found at one or more distant sites from the originating tumor. Anticancer activity may further encompass reduction, complete dissolution, or inhibition of growth of localized or disseminated tumors comprising homogenous or heterogeneous populations of cancer cells.

(2) Anti angiogenic activity is defined as the capacity to inhibit angiogenesis or blood vessel formation. The involvement and recruitment of vascular endothelial cells and expression of pro-angiogenic genes such as vascular endothelial growth factor (VEGF) by tumor cells is a phenomenon well known to those skilled in the art. Specific targeting of angiogenesis promoting factors or vascular endothelial cells is a recognized methodology of inhibiting growth of cancer cells and tumors and targeting them for destruction and/or inhibition of proliferation and/or promotion of differentiation.

(3) Antimetastatic activity is defined as the destruction and/or inhibition of proliferation and/or promotion of differentiation of cancer cells that have the capacity to move away form the site of an initial tumor and may be found at one or more distant sites from the originating tumor, and/or the inhibition of one or more process involved in invasion and dispersal (such as attachment of cancer cells to blood vessel walls and subsequent penetration into tissues). Such cancer cells may be in the form of isolated single cells present in the circulatory system of a subject including a human or non-human animal. Such cells may also include but is not limited to cancers such as lymphomas, leukemias or other malignant forms of circulating cells which may not originate from a specific tumor site and may be of a disseminated nature.

(4) Enhancers of the effect of radiation may be defined as the capacity of another type of therapy to increase the anticancer activity of various forms of radiotherapy. The enhancement may be further defined as an increase in cancer cell or tumor destruction and/or inhibition of proliferation and/or promotion of differentiation and/or inhibition of tumor growth or metastasis observed relative to that achieved when radiation therapy is utilized alone for treatment. The enhancement of the effect of radiation may result either when radiation therapy is performed before, after or in conjunction with adenoviral therapy. In addition, the radiation therapy may enhance the activity of the viral therapy, for example, but not by way of limitation, by increasing promoter activity driving gene expression or increasing effective gene product levels of an additional therapeutic gene.

(5) Enhancers of the effect of chemotherapy may be defined as the capacity of another type of therapy to increase the anticancer activity of various forms of chemotherapy. The enhancement may be further defined as an increase in cancer cell or tumor destruction and/or inhibition of proliferation and/or promotion of differentiation and/or inhibition of tumor growth or metastasis observed relative to that achieved when chemotherapy is utilized alone for treatment. The enhancement of the effect of chemotherapy may result either when chemotherapy is performed before, after or in conjunction with the other type of therapy. In addition, chemotherapy may enhance the activity of viral therapy, for example, but not by way of limitation, by increasing promoter activity driving gene expression or increasing effective gene product levels of an additional therapeutic gene.

(6) Promotion of immunity may be defined as an enhanced therapeutic effect caused by direct or indirect enhanced expression of a gene or genes causing an immune response in a subject including a human or non-human animal. The enhanced immune response may result in but is not limited to an initiation or enhancement of an anticancer, anti-allergic, anti-inflammatory, anti-bacterial or anti-viral response in a subject including a human or non-human animal. Further, the induced or enhanced immune response may further promote the activity of the initial therapy or another form of therapy, for example, but not by way of limitation, by increasing promoter activity driving gene expression or increasing effective gene product levels of an additional therapeutic gene.

### 6. EXAMPLE 1

### 6.1 Materials and Methods

Construction of Bipartite Conditionally Replication Competent Terminator Adenoviruses. A bipartite adenovirus permits simultaneous expression of two genes from a single adenovirus. To construct such a virus the AdenoQuick cloning system from OD 260 Inc (Boise, Id.) is employed. This system utilizes two shuttle vectors (pE1.2 and pE3.1) in which the transgenes must be inserted before being transferred into a large adenoviral plasmid rescue vector (e.g. pAd, FIG. 4). The E1A region has been deleted from pAd leaving the E1B region intact. The expression cassette in which the PEG-Prom drives Early Region 1A (E1A) of the adenovirus is inserted into the multiple cloning site (MCS) of pE1.2. The other expression cassette, in which the CMV promoter drives expression of a gene of interest e.g., IFN-γ is inserted into the MCS of pE3.1. In both shuttle plasmids the MCS is flanked by two sets of restriction sites. Selective cloning is achieved because sticky ends generated by restriction digestion are incompatible with sites generated in the two different vectors (GAG vs. AGA in pE1.2; CCA vs. ATG in pE3.1). The pAd vector has two pairs of SfiI sites, one in the E1 region the other in the E3 region. The SfiI sites at the E1 region generate sticky ends that are incompatible with each other but are complementary with those generated by digesting pE1.2 with AlwNI, BstAPI, DraIII or PflMI. The SfiI sites at the E3 region generate sticky ends that are incompatible with each other and with those present in the E1 region but are compatible with those generated by digesting plasmid pE3.1 with AlwNI, BstAPI, DraIII or PflMI. Expression cassettes ligated to the respective shuttle plasmids pE1.2 and pE3.1 are released by digestion and ligated to SfiI digested pAd in a four-fragment ligation. The ligation product is transformed into E. coli and clones selected for resistance to ampicillin (ampicillin resistance gene provided by pAd) and kanamycin (kanamycin resistance gene provided by the fragment from the shuttle vector). Cosmid DNA is amplified by standard large scale preparation using Cesium chloride density gradient ultracentrifugation, digested with PacI restriction enzyme and transfected into HEK293 cells for in vivo recombination. HEK293 cells are human embryonic kidney cells that contain and express the essential E1 region of the viral genome. This complementation, which is necessary because E1 is deleted in the vectors, does not occur in other cell types. This is an added safety feature for gene therapy purposes.

Construction of Recombinant Infectivity Enhanced Triage Adenoviruses. To construct infectivity enhanced recombinant adenoviruses, recombination between a “rescue” plasmid containing an almost complete copy of the viral genome and a “shuttle” plasmid containing a foreign (or modified viral) gene flanked by surrounding regions of the adenovirus genome is utilized. Upon co-transfection and recombination between these two plasmids, a recombinant viral genome is generated. The DNA sequence encoding pK7, RGD or potentially any other type of capsid modification is cloned into the shuttle plasmid e.g. pNEB.PK.Pk7 or similar vector containing the fiber sequence (Dmitriev et al. (1998) J Virol 72:9706-9713; Blackwell et al. (2000) Hum Gene Ther 11:1657-1669). The wild type fiber of the adenoviral vector is replaced with the modified fiber by homologous recombination in bacteria (Dmitriev et al. (1998) J Virol 72:9706-9713; Blackwell et al. (2000) Hum Gene Ther 11:1657-1669). After homologous recombination, the genome of the new adenoviral vector is released from the plasmid backbone by digestion with restriction enzyme digestion e.g. PacI. The obtained plasmid is then utilized for transfection of 293 cells to rescue the virus. The pK7, RGD or other alternative modification in the virus is confirmed by PCR as well as by cycle sequencing of viral DNA isolated from CsCl-purified virions. To construct a Triage Virus one shuttle plasmid contains a cassette in which the expression of the E1A gene, necessary for adenoviral replication, is under the control of PEG-3 promoter while the other plasmid contains a cassette in which a therapeutic gene of interest whose expression is controlled by the CMV promoter or other suitable promoter. In the first step a plasmid is derived by homologous recombination between an adenovirus with a fiber modification constructed as described supra and a shuttle plasmid containing the E1A region under the control of the PEG-3 promoter as described for the Terminator Virus to generate a conditionally replicative infectivity enhanced adenovirus. Subsequently, a vector encoding a therapeutic gene of interest is derived by homologous recombination between shuttle plasmids encoding the gene of interest under the control of CMV or other promoter and the conditionally replicative infectivity enhanced virus comprising fiber modification and PEG-promoter driven E1A expression as described for the Terminator Virus. The recombinant plasmid containing the adenoviral genome encoding modified fiber, PEG-3 promoter driven E1A and E1B and an E3 region containing a gene of interest driven by a heterologous promoter, is amplified by standard large scale preparation using a CsCl gradient and transfected into a human cancer cell line such as DU-145 or HeLa showing high activity of the PEG-3 promoter. Activity of the PEG-3 promoter (Su et al. (1999) Proc Natl Acad Sci USA 96(26):15115-151120; Su et al. (2002) J Cell Physiol 192(1):34-44) in transformed cells drives viral replication and enables production of Triage Viruses.

Administration of Recombinant Conditionally Replicative Adenoviruses to Animals, AsPC-1 cells were used to establish tumor xenografts in athymic nude mice. 2×10⁶ cells were injected subcutaneously in both the right and left flanks of each mouse. After the establishment of visible tumors of ˜75 mm³, requiring ˜4-5 days, intratumoral injections of different Ads were given only to the tumor on the left flank at a dose of 1×10⁸ pfu in 100 μl. The injections were given three times a week for the first week and then twice a week for two more weeks to a total of seven injections. The tumor size was measured by a caliper and the tumor volume was determined using the formula π/6×(large diameter)×(small diameter)². The experiment was stopped after 4 weeks because with Terminator Virus injections the tumors were either completely or almost completely eradicated. The tumors were removed and the tumor weight was determined.

Fluorescence Activated Cell Sorting (FACS) Analysis for Apoptosis and Necrosis (Annexin-V-Binding Assay). Cells were trypsinized and washed once with complete media. Aliquots of cells (5×10⁵) were resuspended in complete media (0.5 ml) and stained with FITC-labeled Annexin-V (kit from Oncogene Research Products, Boston, Mass.) according to the manufacturer's instructions. Propidium iodide (PI) was added to the samples after staining with Annexin-V to exclude late apoptotic and necrotic cells. Flow cytometry was performed immediately after staining.

### 6.2 Results and Discussion

PEG-3 Promoter Driven Terminator Virus Inhibits Growth of Pancreatic Cancer Cells but not Normal Cells In Vitro. Four human pancreatic cancer cell lines, MIA Paca-2, PANC-1, AsPC-1 and BxPC-3 and two normal cells, FM-516-SV, immortal normal human melanocytes and IM-PHFA, immortal primary human fetal astrocytes were utilized in this working example. The cells were either uninfected or infected with Ad.vec (control empty virus) or different transgene expressing adenoviruses at an m.o.i. of 100 pfu/cell and cell viability was analyzed by standard MTT assay over a period of 6 days. Infection with only Ad.CMV-E1A and Ad.CMV-E1A-IFN-γ resulted in profound growth inhibition of FM-516 and IM-PHFA cells. Infection with Ad.PEG-E1A, Ad.CMV-IFN-γ, Ad.PEG-IFN-γ and Ad.PEG-E1A-IFN-γ resulted in little to no growth inhibition in comparison to control or Ad.vec infected cells. In contrast, in all the pancreatic cancer cells, both Ad.CMV-E1A-E1A-IFN-γ and Ad.PEG-E1A-IFN-γ (Terminator Virus) as well as Ad.CMV-E1A and Ad.PEG-E1A infection resulted in profound growth inhibition in comparison to the control or Ad.vec infected cells. Infection with Ad.CMV-IFN-γ and Ad.PEG-IFN-γ resulted in ˜50% growth inhibition in comparison with control or Ad.vec-infected cells. These findings indicate that the PEG-Prom allows adenoviral replication specifically in cancer cells, protecting normal cells from growth inhibition because of adenoviral replication.

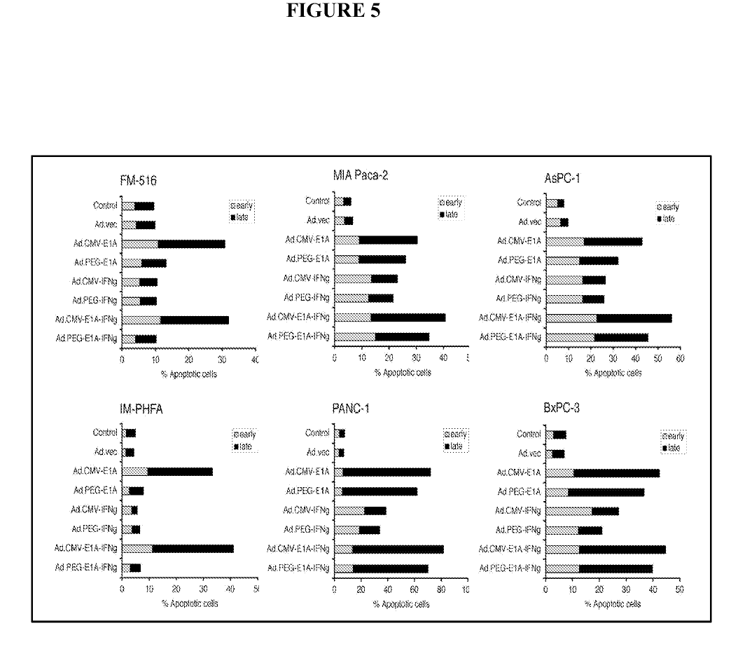

Annexin V staining and analysis by flow cytometry confirmed the growth inhibition (FIG. 5). Annexin V staining allows differentiation between apoptotic and necrotic cells. As shown in FIG. 5, infection with only Ad.CMV-E1A and Ad.CMV-E1A-IFN-γ, and not any other treatment regimen, resulted in significant percentage of early apoptotic and late apoptotic (necrotic) cells in FM-516 and IM-PHFA cells. However, all of the adenoviruses, except for Ad.vec, resulted in significant apoptosis in the pancreatic cancer cell lines. Infection with the replication competent adenovirus resulted predominantly in necrosis evidenced by increase in late apoptotic cells while infection with Ad.CMV-IFN-γ and Ad.PEG-IFN-γ resulted predominantly in apoptosis as evidenced by increase in early apoptotic cells.

Gamma-radiation or DNA damaging agents stimulate PEG-3 promoter activity (Su et al. (1999) Proc Natl Acad Sci USA 96(26):15115-151120; Su et al. (2002) J Cell Physiol 192(1):34-44). Therefore, utilization of a Terminator Virus in conjunction with radio- or chemotherapy therapy may result in both enhanced viral replication and the resultant enhanced expression level of the gene of interest encoded by the Terminator Virus in the E3 region. Thus dual treatment with a radio- or chemotherapeutic agent and a Terminator Virus may result in enhanced overall activity both of viral replication and expression of the exogenous gene of interest. In addition, specific targeting or enhanced infectivity may be obtained by utilizing an appropriate Triage Virus in conjunction with radio- or chemotherapy.

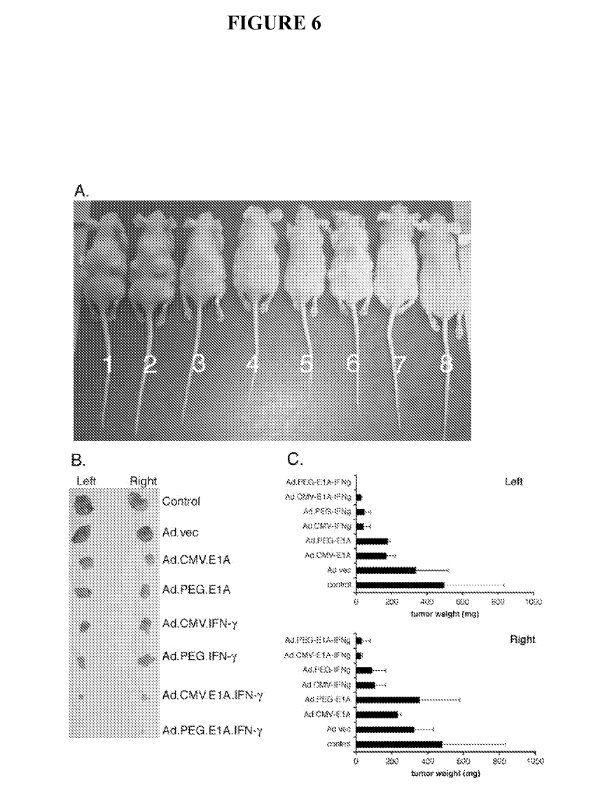

Terminator Virus Treatment Inhibits the Growth of Pancreatic Cancer Cell Xenografts in Athymic Nude Mice. In vitro findings (FIG. 5) were tested further in animal studies (FIG. 6). Eight sets of mice were injected with AsPC-1 cells to establish tumor xenografts and treated as described in 6.1.3. The animals were injected with cells on both flanks but treated with adenoviruses only on the left side, while the right sides were left untreated. While Ad.CMV-E1A or Ad.PEG-E1A inhibited the growth of the tumors on the left side, they had some inhibitory effect on the tumors on the right side, which was not statistically significant. On the other hand injection with Ad.CMV-IFN-γ or Ad.PEG-IFN-γ (Terminator Virus) resulted in complete to nearly complete eradication of the tumor both on the left and right sides (mouse numbers 7 and 8, FIG. 5A; corresponding tumor volume FIGS. 5B and 5C). These findings indicate that Ad.CMV-E1A-IFN-γ or Ad.PEG-E1A-IFN-γ (Terminator Virus) display potent inhibitory effects on the growth of the xenografts, which is due to the profound effect of viral replication as well as stimulation of anti-tumor immunity by the production of bursts of IFN-γ.

Tropism Modification of Adenovirus Improves Infection of Adenovirus in Prostate Cancer Cells. The absence of the primary adenoviral receptor, i.e. the Coxsackie-Adenovirus Receptor (CAR), in target cells is a substantial obstacle to effective gene therapy, as it limits the access of cells to therapeutic virus. To overcome this obstacle, adenoviral vectors may be targeted to alternative cellular receptors by genetically modifying surface properties of the viral capsid. As working examples of this methodology, the effects of three genetic modifications in the adenoviral fiber capsid on transgene [luciferase (LUC) and green fluorescent protein (GFP)] expression in SV40 T antigen immortalized normal human prostate epithelial cells P69, and DU-145 and PC-3 prostate carcinoma cells were determined. Adenoviruses were constructed that express both LUC and GFP in either a wild-type background (Ad.GFP.LUC) or in a genetically modified background. The genetic modifications included insertion of an Arg-Gly-Asp (RGD)-containing peptide (permitting attachment and entry through integrin receptors) (Ad.RGD.GFP.LUC), a poly lysine (pK7)-peptide (GSGSGSGSGSKKKKKKK) (SEQ ID NO:4) (permitting attachment and entry through heparin sulfate-containing receptors) (Ad.pK7.GFP.LUC) and both RGD and pK7 peptides (Ad.RGD.pK7.GFP.LUC). The expression of GFP was analyzed by FACS. In P69 cells all the three tropism modified Ads showed increased infectivity compared to Ad.GFP.LUC (FIG. 7A). In DU-145 and PC-3 cells Ad.RGD.GFP.LUC and Ad.GFP.LUC showed similar levels of infectivity (FIG. 7BC). However both Ad.pK7.GFP.LUC and Ad.RGD.pK7.GFP.LUC showed higher levels of infectivity in the prostate cancer cells. The combination of RGD and pK7 modifications had similar effects as compared to only the pK7 modification indicating that in prostate cancer cells this particular modification alone may be sufficient to facilitate higher levels of infection. Similar findings were also observed when the infectivity was analyzed by luciferase reporter assays. These findings indicate that specific modifications in the adenoviral capsid fiber can improve infectivity of prostate tumor cells by Triage viruses.

### 7. EXAMPLE 2

### 7.1 Materials and Methods

Cell lines, culture conditions and viability assays. DU-145, PC-3 and LNCaP prostate cancer cells were obtained from the ATCC and cultured as described (Lebedeva et al. (2003) Oncogene 22:8758-73). The generation and characterization of DU-145-Bcl-x_{L}, PC-3-Bcl-x_{L} and LNCaP-Bcl-2 have been described previously (Id.). P69 cells are normal human prostate epithelial cells immortalized by SV40 T/t Ag and are cultured as described (Su et al. (2005) Oncogene 24:7552-66). Cell viability was determined by standard MTT assays (Lebedeva et al. (2002) Oncogene 21:708-18).

Construction of a Conditional Replication Competent Adenovirus (CRCA). To construct the CRCA Ad.PEG-E1A-mda-7, a CTV, the AdenoQuick cloning system from OD 260 Inc (Boise, Id.) was employed (Sarkar et al. (2005) Proc Natl Acad Sci USA 102:14034-9; Sarkar et al. (2005) Cancer Res 65:9056-63). This system utilizes two shuttle vectors, pE1.2 and pE3.1, in which the transgene cassettes PEG-Prom driving E1A and CMV promoter driving mda-7/IL-24 were inserted, respectively, before being transferred into a large adenoviral plasmid (pAd). Adenoviral amplification, purification, titration and infection were performed as described. Similar strategies were used to generate Ad.CMV-E1A-mda-7. Ad.CMV-mda-7 and Ad.PEG-mda-7 were constructed as previously described.

Annexin V binding assay. Annexin V binding assays were performed as described (Lebedeva et al. (2003) Oncogene 22:8758-73).

Preparation of Whole Cell Lysates and Western Blot Analyses. Preparation of Whole Cell Lysates and Western Blot Analyses was Performed as Described (Sarkar et al. (2005) Proc Natl Acad Sci USA 102:14034-9). The primary antibodies used were anti-E1A (1:1000; mouse monoclonal; Upstate Biotechnology, Waltham, Mass.), anti-MDA-7 (1:2000; rabbit polyclonal), anti-EF1α (1:1000; mouse monoclonal; Upstate).

Human prostate cancer xenografts in athymic nude mice. DU-145-Bcl-x_{L} cells (2×10⁶) were injected subcutaneously in 100 μl of PBS in both flanks of male athymic nude mice (NCR^{nu/nu}; 4 weeks old; ˜20 g body weight) (Sarkar et al. (2005) Proc Natl Acad Sci USA 102:14034-9, Sarkar et al. (2005) Cancer Res 65:9056-63). After establishment of visible tumors of ˜75 mm³, requiring ˜4-5 days, intratumoral injections of different Ads were given only to the tumors on the left flank at a dose of 1×10⁸ pfu in 100 μl. No injection was given to the right-sided tumors. The injections were given 3 times a week for the first week and then twice a week for two more weeks for a total of seven injections. A minimum of 5 animals was used per experimental point. Tumor volume was measured twice weekly with a caliper and calculated using the formula π/6×larger diameter×(smaller diameter)². At the end of the experiment the animals were sacrificed and the tumors were removed and weighed.

Immunofluorescence analysis. Tumors were harvested from the animals, fixed in formalin and embedded in paraffin. The sections were deparaffinized and were permeabilized with 0.1% TritonX-100 in PBS for 30 minutes. Sections were then blocked for 1 h at room temperature with 2% goat serum and 1% BSA in PBS and incubated with anti-E1A antibody (1:100) or anti-MDA-7 antibody (1:100) overnight at 4° C. Sections were then rinsed in PBS, and incubated with Alexa488-conjugated anti-mouse or anti-rabbit IgG (Molecular Probes), respectively for 1 h at room temperature. The sections were mounted in VectaShield fluorescence mounting medium containing 4,6-diamidino-2-phenylindole (Vector Laboratories). For CD31 staining, FITC-conjugated rat anti-mouse CD31 monoclonal antibody (BD Pharmingen) was used. A confocal laser scanning microscope analyzed the images.

Statistical analysis. Statistical analysis was performed using one-way analysis of variance (ANOVA), followed by Fisher's protected least significant difference analysis. A p value of <0.05 was considered significant.

### 7.2 Results

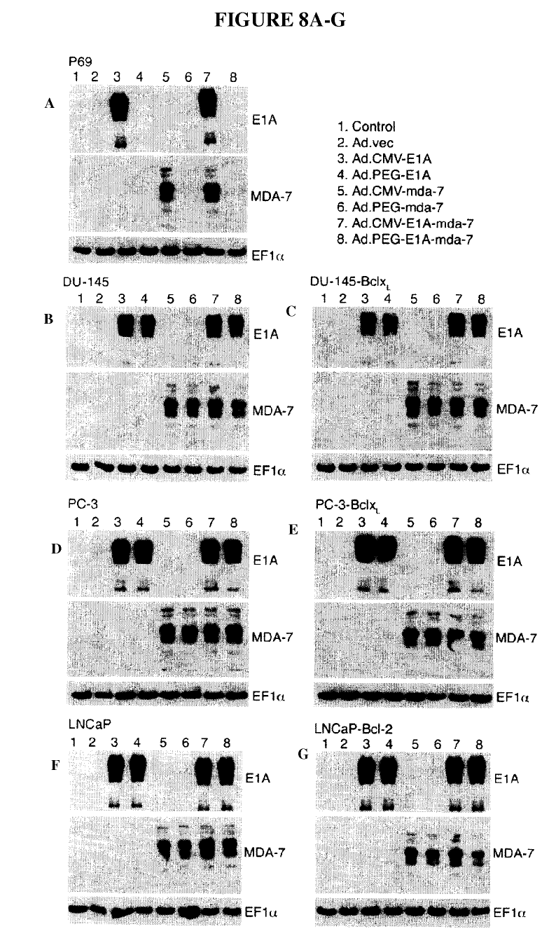

The PEG-3 promoter (PEG-Prom) targets selective expression of Ad E1A and MDA-7/IL-24 in prostate cancer cells. Apart from Ad.PEG-E1A-mda-7 (CTV), a series of additional Ads were created, such as Ad.CMV-E1A-mda-7, in which viral replication is controlled by the CMV promoter and which also expresses mda-7/IL-24, and Ad.CMV-E1A and Ad.PEG-E1A, in which viral replication is controlled by the CMV promoter or the PEG-Prom, respectively, to compare and contrast their relative efficacy. We also employed Ad.CMV-mda-7 and Ad.PEG-mda-7, replication-incompetent Ad in which the CMV or the PEG promoter drives mda-7/IL-24 expression, respectively. A replication-incompetent empty Ad, Ad. vec was used as a control. Experiments were performed in three prostate cancer cell lines, androgen-nonresponsive DU-145 and PC-3 cells and androgen-responsive LNCaP cells and their Ad.mda-7-resistant variants, namely DU-145-Bcl-x_{L}, PC-3-Bcl-x_{L} and LNCaP-Bcl-2. These Bcl-2 or Bcl-x_{L} overexpressing clones and their resistance to Ad.mda-7 have been extensively characterized in previous studies (Lebedeva et al. (2003) Oncogene 22:8758-73). As a control P69, normal prostate epithelial cells immortalized by SV40T/t Ag, was employed. The replication-incompetent Ads were infected at an m.o.i. of 5000 vp/cell while the replication-competent Ads were infected at 1000 vp/cell. The functionality of these constructs was ascertained following Ad infection by monitoring protein levels of MDA-7/IL-24 and E1A, a marker for adenoviral replication, by Western blot analysis after appropriate viral infection (FIG. 8A-G). Western blot analysis detects multiple E1A gene products ranging from 36-50-kDa and multiple glycosylated forms of MDA-7/IL-24 protein ranging from 21-28-kDa (Sarkar et al. (2005) Proc Natl Acad Sci USA 102:14034-9, Sarkar et al. (2005) Cancer Res 65:9056-63).

Infection of normal immortal human P69 prostate epithelial cells with Ad.CMV-E1A or Ad.CMV-E1A-mda-7 but not Ad.PEG-E1A or Ad.PEG-E1A-mda-7 (CTV) resulted in production of E1A proteins, while in prostate carcinoma cells infection with all four replication-competent Ads generated E1A proteins (FIG. 8A-G). No E1A proteins were detected in any cell line following infection with replication-incompetent Ads. In P69 cells, infection with Ad.CMV-E1A-mda-7 and Ad.CMV-mda-7 resulted in MDA-7/IL-24 production while infection with Ad.PEG-mda-7 or Ad.PEG-E1A-mda-7 (CTV) resulted in barely detectable levels of MDA-7/IL-24 production (FIG. 8A-G). In prostate cancer cells, infection with Ad.CMV-mda-7, Ad.PEG-mda-7, Ad.CMV-E1A-mda-7 or Ad.PEG-E1A-mda-7 (CTP) generated significant MDA-7/IL-24 production. No MDA-7/IL-24 protein production could be detected in control uninfected cells or following infection with Ad.vec, Ad.CMV-E1A or Ad.PEG-E1A. These findings document that the PEG-Prom facilitates cancer cell-selective replication of Ad and mda-7/IL-24 expression.

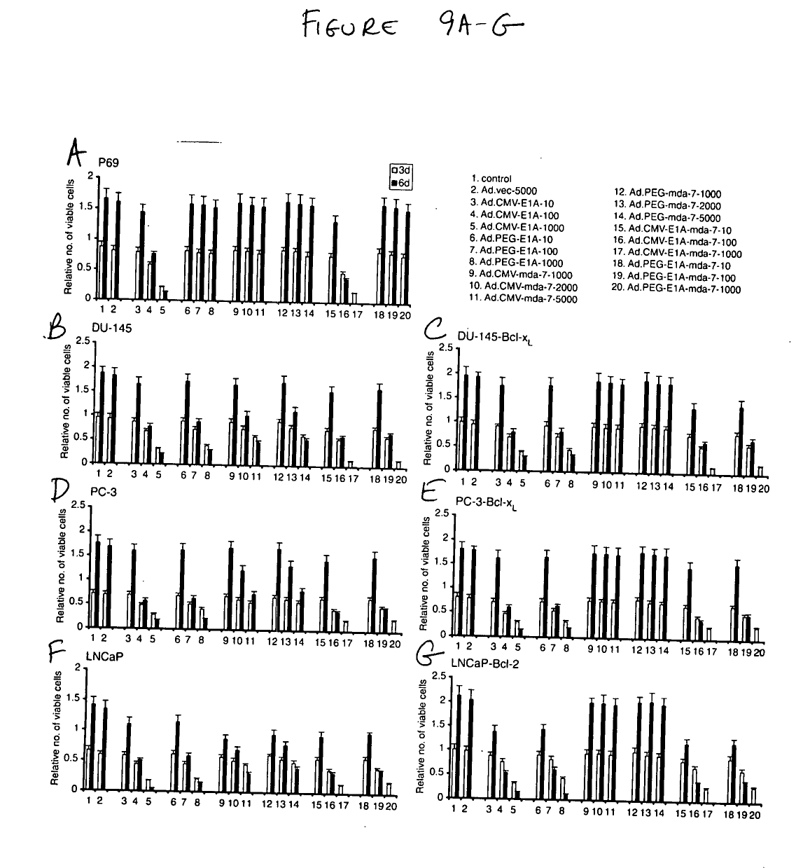

Ad.PEG-E1A-mda-7 (CTP) selectively kills prostate cancer cells, without harming normal prostate cells. The effects of the engineered Ads on cell viability and apoptosis were evaluated in the various prostate cell lines. Cells were infected with replication-competent Ads at 10, 100 and 1000 vp/cell and with replication-incompetent Ads at 1000, 2000 and 5000 vp/cell. In P69 cells, infection with only Ad.CMV-E1A or Ad.CMV-E1A-mda-7, but not with Ad.PEG-E1A, Ad.CMV-mda-7, Ad.PEG-mda-7 or Ad.PEG-E1A-mda-7 (CTV), induced profound growth inhibition (FIG. 9A-G). In contrast, in all prostate cancer cells, both parental and mda-7/IL-24-resistant, Ad.CMV-E1A-mda-7, Ad.PEG-E1A-mda-7 (CTV), Ad.CMV-E1A and Ad.PEG-E1A infection resulted in significant growth inhibition. Infection with Ad.CMV-mda-7 and Ad.PEG-mda-7 inhibited growth of parental DU-145, PC-3 and LNCaP cells, but not their resistant counterparts. It should be noted that the level of growth inhibition observed with 5000 vp/cell of Ad.CMV-mda-7 and Ad.PEG-mda-7 was equivalent to that observed with only 100 vp/cell of replication-competent Ad indicating that the replication-competent Ads are much more potent than the replication-incompetent Ads in cell growth inhibition. These findings indicate that the PEG-Prom allows Ad replication specifically in cancer cells, protecting normal cells from growth inhibition because of Ad replication. The observation that mda-7/IL-24 exerted no direct growth inhibitory effect on normal cells confirms the cancer cell-selectivity of this therapeutic approach. Most importantly, Ad.PEG-E1A-mda-7 was able to overcome the resistance of Bcl-2 and Bcl-x_{L} overexpressing clones to mda-7/IL-24 indicating its potential therapeutic application in prostate cancer patients frequently showing Bcl-2 and Bcl-x_{L} overexpression.

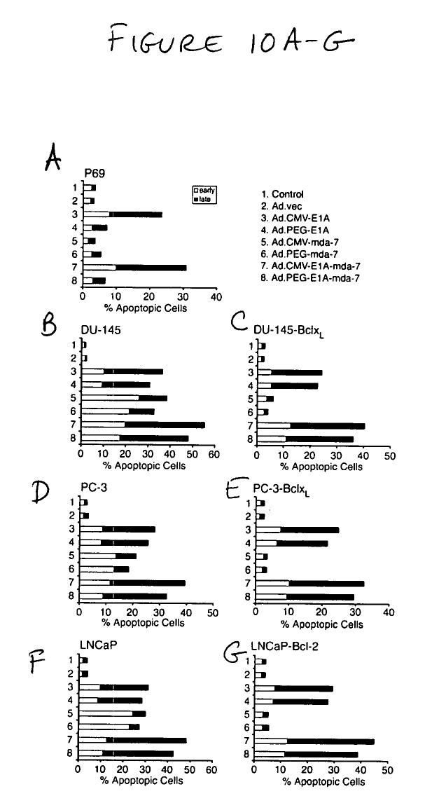

Ad.PEG-E1A-mda-7 (CTV) selectively induces apoptosis in prostate cancer cells. To investigate the mechanism of growth inhibition, Annexin V staining assays, which permit differentiation between apoptotic and necrotic cells, were performed (FIG. 10A-G). Infection with only Ad.CMV-E1A and Ad.CMV-E1A-mda-7 (CTV) elevated the percentage of early apoptotic and late apoptotic (necrotic) P69 cells. However, all of the Ads, except for Ad.vec, resulted in significant apoptosis in DU-145, PC-3 and LNCaP parental prostate cancer cells. Infection with the replication competent Ads resulted predominantly in necrosis as manifested by an increase in late apoptotic cells, while infection with Ad.CMV-mda-7 and Ad.PEG-mda-7 resulted in predominantly apoptosis as evidenced by an increase in early apoptotic cells. Although Ad.CMV-mda-7 and Ad.PEG-mda-7 had no apoptotic effect on DU-145-Bcl-x_{L}, PC-3-Bcl-x_{L} and LNCaP-Bcl-2 cells, Ad.CMV-E1A-mda-7 and Ad.PEG-E1A-mda-7 (CTV) could override this resistance and induce significant apoptosis and necrosis in these cells. It is important to mention that while the replication-incompetent Ads were used at 5000 vp/cell, replication-competent Ads were used at 1000 vp/cell.

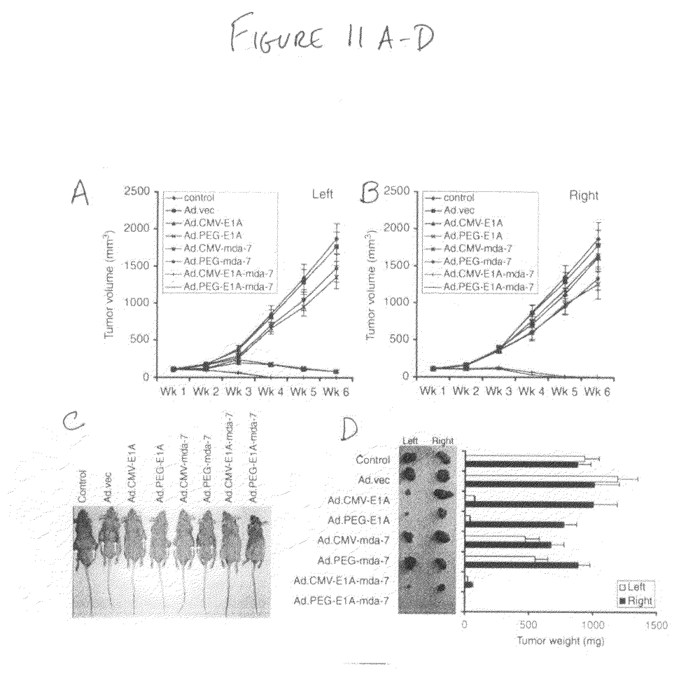

Ad.PEG-E1A-mda-7 (CTV) eradicates both primary and distant Bcl-X_{L} overexpressing prostate tumors in nude mice. To expand on the in vitro studies, in vivo assays were performed using nude mice containing established DU-145-Bcl-x_{L} subcutaneous xenografts on both right and left flanks. After palpable tumors of ˜75 mm³ developed, in ˜4-5 days, seven intratumoral injections with different Ads, 3× per week for the first week and 2× per week for an additional two weeks, were administered to the tumors on the left flank at a dose of 1×10¹⁰ vp in 100 μl. No injections were given to the right-sided tumors. The experiment was terminated after 6 weeks with injections of Ad.CMV-E1A-mda-7 or Ad.PEG-E1A-mda-7 (CTV) since tumors on both sides showed regression after only three injections and with seven injections they were completely eradicated (FIG. 11A-D). Further studies are required to determine if a single or double injection with the CTV elicits any discernible anti-tumor activity in this animal model. Additionally both control and Ad.vec-infected DU-145-Bcl-x_{L} tumor xenografts reached tumor volumes of ˜2000 mm³ requiring them to be sacrificed. While Ad.CMV-E1A or Ad.PEG-E1A inhibited the growth of tumors on the left flank they had some inhibitory effect on tumors on the right side, which was not statistically significant. Ad.CMV-mda-7 or Ad.PEG-mda-7 also displayed marginal effects on the growth of both left- and right-sided tumors, which correlates with the in vitro findings of resistance of DU-145-Bcl-x_{L} cells to mda-7/IL-24. The observation that intratumoral injection of Ad.PEG-E1A-mda-7 (CTV) completely eradicated the primary and the distant tumor (comparable to a metastasis) provides confidence that this strategy may prove amenable for successfully treating aggressive cancers.

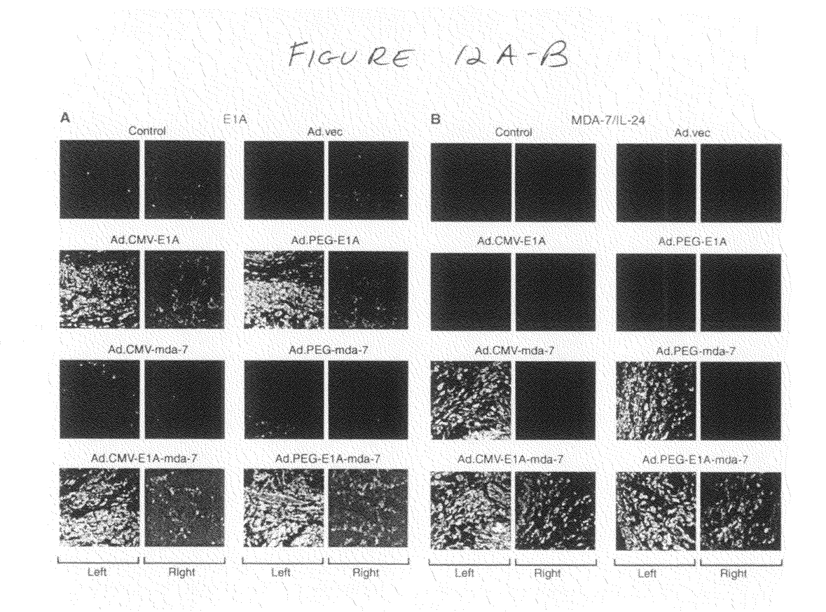

Ad.PEG-E1A-mda-7 (CTV) replicates at distant tumor sites and generates MDA-7/IL-24 protein. Since Ad.CMV-E1A-mda-7 or Ad.PEG-E1A-mda-7 (CTV) eradicated both the left-sided injected and right-sided uninjected tumors, we analyzed the replication efficiency and transgene delivery by these Ads (FIG. 12A-B). Tumors were harvested from the animals after 3 injections and formalin-fixed paraffin embedded sections were stained for E1A and MDA-7/IL-24 and analyzed by a confocal laser scanning microscopy. The replication competent Ads, and not the replication-incompetent Ads, could effectively replicate in the left-sided tumors as evidenced by robust staining for E1A protein (FIG. 12A-B). Interestingly, staining for E1A could also be detected in the right-sided tumors, albeit at a much lower level than their left-sided counterparts, indicating that the Ad could enter into the circulation and replicate in the right-sided tumor cells. Staining for MDA-7/IL-24 supported these findings (FIG. 12A-B). While all of the mda-7/IL-24 expressing Ads effectively generated MDA-7/IL-24 protein in the left-sided tumors, only Ad.CMV-E1A-mda-7 and Ad.PEG-E1A-mda-7 (CTV), and not Ad.CMV-mda-7 and Ad.PEG-mda-7, generated MDA-7/IL-24 protein in the right-sided tumors. These data indicate that the combination of Ad replication and robust MDA-7/IL-24 generation resulted in a significant response that could effectively eliminate both the primary and distant tumor.

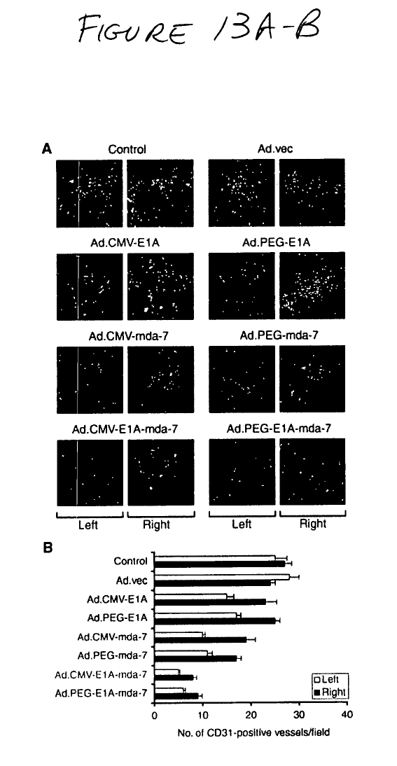

Ad.PEG-E1A.mda-7 (CTV) inhibits angiogenesis. MDA-7/IL-24, as a secreted cytokine is known to inhibit angiogenesis (Tong et al. (2005) Mol Ther 11: 160-72). Based on this consideration, we analyzed the microvessel density in the tumor by staining for CD31 (FIG. 13). After 3 injections, Ad.CMV-E1A and Ad.PEG-E1A showed marginal effects on angiogenesis inhibition on the left-sided tumors with no effect on the right-sided uninjected tumors. Ad.CMV-mda-7 and Ad.PEG-mda-7 significantly inhibited angiogenesis in both left- and right-sided tumors. Since these two Ads did not impact on the growth of the tumors it might be inferred that inhibition of angiogenesis alone may not be sufficient to inhibit the growth of the tumors and inhibition of growth of the cancer cells themselves is mandatory to provoke an enduring anti-tumor effect. Ad.CMV-E1A-mda-7 and Ad.PEG-E1A-mda-7 (CTV) profoundly inhibited angiogenesis in both left- and right-sided tumors indicating that the MDA-7/IL-24 protein generated exhibits its complete spectrum of biological activities (FIG. 13).

Various publications are cited herein, which are hereby incorporated by reference in their entireties.

What is claimed is:

1. An adenovirus comprising a PEG-3 promoter operably linked to the E1A gene, further comprising a heterologous gene of interest, operably linked to a promoter.

2. The adenovirus of claim 1, wherein the gene of interest has anti-cancer activity.

3. The adenovirus of claim 1, wherein the gene of interest promotes an anti-angiogenic effect.

4. The adenovirus of claim 1, wherein the gene of interest promotes an anti-metastatic effect.

5. The adenovirus of claim 1, wherein the gene of interest enhances the effect of radiation therapy.

6. The adenovirus of claim 1, wherein the gene of interest enhances the effect of chemotherapy.

7. The adenovirus of claim 1, wherein the gene of interest is mda-7.

8. A method of inhibiting the proliferation of a cancer cell, comprising infecting said cell with the adenovirus of claim 1, wherein infection is of a cancer cell derived from a cancer selected from the group consisting of a nasopharyngeal tumor, a thyroid tumor, a central nervous system tumor, melanoma, a vascular tumor, a blood vessel tumor, an epithelial tumor, a non-epithelial tumor, leukemia, lymphoma, a cervical cancer, a breast cancer, a lung cancer, a prostate cancer, a colon cancer, a hepatic carcinoma, a urogenital cancer, an ovarian cancer, a testicular carcinoma, an osteosarcoma, a chondrosarcoma, a gastric cancer, or a pancreatic cancer.

9. A method of treating a subject suffering from a cancer, where the cancer is selected from the group consisting of breast cancer, lung cancer, prostate cancer, colon cancer, rectal cancer, hepatic carcinoma, urogenital cancer, ovarian cancer, testicular carcinoma, osteosarcoma, chondrosarcoma, gastric cancer, pancreatic cancer, nasopharyngeal cancer, thyroid cancer, neuroblastoma, astrocytoma, glioblastoma multiforme, melanoma, hemangiosarcoma, an epithelial cancer, a non-epithelial cancer such as squamous cell carcinoma, leukemia, lymphoma, and cervical cancer, comprising administering, to the subject, an effective amount of a modified adenovirus according to claim 1.

10. A method of treating a subject suffering from a refractory cancer comprising administering, to the subject, an effective amount of a modified adenovirus according to claim 7.

11. The adenovirus of claim 1, wherein the gene of interest promotes immunity.

12. The adenovirus of claim 1, wherein the gene of interest promotes an anti-inflammatory effect.

13. The adenovirus of claim 1, wherein the gene of interest promotes an anti-viral effect.

14. The adenovirus of claim 1, wherein the gene of interest is operably linked to constitutively active promoter.

15. The adenovirus of claim 1, wherein the gene of interest is operably linked to an inducible promoter.

16. The adenovirus of claim 1, wherein the gene of interest is operably linked to tissue specific promoter.

17. The adenovirus of claim 1 wherein the gene of interest is a secreted gene product.

18. The adenovirus of claim 17, wherein the secreted gene of interest is active at a site distant from its site of expression.

19. The adenovirus of claim 17, wherein the secreted gene of interest has anti-cancer activity.

20. The adenovirus of claim 17, wherein the secreted gene of interest promotes immunity.

21. The adenovirus of claim 17, wherein the secreted gene of interest promotes an anti-angiogenic effect.

22. The adenovirus of claim 17, wherein the secreted gene of interest promotes an anti-metastatic effect.

23. The adenovirus of claim 17, wherein the secreted gene of interest promotes an anti-inflammatory effect.

24. The adenovirus of claim 17, wherein the secreted gene of interest promotes an anti-viral effect.

25. The adenovirus of claim 17, wherein the secreted gene of interest enhances the effect of radiation therapy.

26. The adenovirus of claim 17, wherein the secreted gene of interest enhances the effect of chemotherapy.

27. The adenovirus of claim 1, wherein a capsid protein is modified to facilitate infection of a cancer cell.

28. The adenovirus of claim 27, wherein a capsid protein modification comprises an RGD peptide, a pK7 peptide or combination of RGD and pK7 peptide.


SEQUENCE LISTING (5 sequences)

SEQ ID NO: 1 - DNA, 2614 bp, Unknown
  Rat (Rattus norvegicus ) genomic DNA, /promoter region
acatgggcacgcgtggtcgacggcccgggctggctgggcaacacgggttcagcccaggtttcatagtaagttccagacactcctggaaaaacaatacaggtccctgacaaaagaaaaaacaaaacaaaggaaacagaaacatgcgtttttaaaaaagaaggaggagactccatgaaggcaggccttgggtggggtcactgcttctctgtacacaggaggagaattgccaagatcttccggacagtgtggactatactgtaagaccctctcaatacagacagactggacaggcatagtgacacatgcctttaatgcctgcagtactcaggaggaggtggcaggtggaacggctgttctttgaggttcaagaccagcgtggactacagagtgagttccaggacaggcagggctacacagaaaaatcctgtctgaaaacaaaacaaaacccagacagacacaccaaaaacagccaagggaccagagagatgggtcagggcctaatcacttgctactctttgcagaggacccaaatttagttcctataaccctccatgagaagcttcacaattgtctctaactcaattccacccgtgttccgacctccatatgcaccagacatgatatactcacacatacgcacaaacacacacacacacacacacacacacacacacacacacacacacacacggaaaacatataaaataaagatttaaaaaatctttttcttttggccggggtgtgtgggagagcatctgagccatctcaccagcccagggtgcagctctttttctttttttcggagctggggaccgaacccagagccttgtgcttgctaggcaagtgctctaccactgagctaaatccccaaccccggagcacgtctttaatcccagaatcaggaggtagaggtaatgagatcccagtgagcccaaggtcagccgagtctacaaagtgagttccaggacagccagaactaatcttggaaaaacaaacaagggctggtgaggtggttcagtagttaagaacactggctgctcttccagaggtcctgagttcattctcagtaaccacatggtggggatctgatgcctgttctggcatgcagatatacatgcagatagtgcactcctacatttaaaaaaaaaagacataaataatattttaaaacattgggcgttttgtcttctaataaaacttcactgctatcttctaataaaaattcactgctagccgcggggtgtggtggccccatacctttaatcccaacaacttgagaggcagaggcaggcggacctttgagtttgaagctagcctggtctacagagtgagttcaagatagccacggatagtcagaaagtcctgtttcgaacctctccccaaccaaatcactcctgtaatcccagcactctggaggcagtagcaggttagtccctgcttctcagagagaggagagagagagagagagagagagagaggagacacacacacacagagacagagaggagagagaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagatagcgctcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccagcctgagggcgacatgaacgcgctggccttgagagcaatccggacccacgatcgcttttggcaaaccgaaccggaccgaaccggacctccagcccccggggtgacgcgcagtcgccggtgagtgggggatggggcggcctttgggggagtgctggggaggactttctttggcgatggaggctaggagagtgttgtgggatctaggggagactggggaggaacccagatttgaggaaacggcactgaaagccggatgctttatttggtccgagagaggagagcccaggtctagtctctacattgaagggcaggggtcctgaactagaactgcagtacttgtacattgctaaataaagagagggactccaggaggagcagcctgggtctaagaggtaggcaggagaaggttttaggggcctgagcacaagcttgaggagagaaaggttattaaaaagccagacgttacaggtctcagaagggctagccagaaactgtggcttggggttaaggaaagggtttaagagtgtgggcttttggttctgaggatgtaggaacgtgaatgttgagagaagaaccaagtggcggagttgggtgtgagcaatgctattaggaatttgaggcagggattcacgctgctgtgactattttttaacaatgactcagtgctgtgacctgatactgtttccagagcgacttctaaacaaattcccccctttct

SEQ ID NO: 2 - DNA, 1970 bp, Unknown
  Rat (Rattus norvegicus ) genomic DNA, /promoter region
acatgggcacgcgtggtcgacggcccgggctggctgggcaacacgggttcagcccaggtttcatagtaagttccagacactcctggaaaaacaatacaggtccctgacaaaagaaaaaacaaaacaaaggaaacagaaacatgcgtttttaaaaaagaaggaggagactccatgaaggcaggccttgggtggggtcactgcttctctgtacacaggaggagaattgccaagatcttccggacagtgtggactatactgtaagaccctctcaatacagacagactggacaggcatagtgacacatgcctttaatgcctgcagtactcaggaggaggtggcaggtggaacggctgttctttgaggttcaagaccagcgtggactacagagtgagttccaggacaggcagggctacacagaaaaatcctgtctgaaaacaaaacaaaacccagacagacacaccaaaaacagccaagggaccagagagatgggtcagggcctaatcacttgctactctttgcagaggacccaaatttagttcctataaccctccatgagaagcttcacaattgtctctaactcaattccacccgtgttccgacctcccatatgcaccagacatgttatactcacacatacgcacaaacacacacacacacacacacacacacacacacacacacacacacacacggaaaacatataaaataaagatttaaaaaatctttttcttttggccggggtgtgtgggagagcatctgagccatctcaccagcccagggtgcacgtctttttctttttttcggagctggggaccgaacccagagccttgtgcttgctaggcaagtgctctaccactgagctaaatccccaaccccggagcacgtctttaatcccagaatcaggaggtagaggtaatgagatcccagtgagcccaaggtcagccgagtctacaaagtgagttccaggacagccagaactaatcttggaaaaacaaacaagggctggtgaggtggttcagtagttaagaacactggctgctcttccagaggtcctgagttcattctcagtaaccacatggtggggatctgatgcctgttctggcatgcagatatacatgcagatagtgcactcctacatttaaaaaaaaaagacataaataatattttaaaacattgggcgttttgtcttctaataaaacttcactgctatcttctaataaaaattcactgctagccgcggggtgtggtgcccccatacctttaatcccaacaacttgagaggcagaggcaggcggacctttgagtttgaagctagcctggtctacagagtgagttcaagatagccacggatagtcagaaagtcctgtttcgaacctctccccaaccaaatcactcctgtaatcccagcactctggaggcagtagcaggttagtccctgcttctcagagagaggagagagagagagagagagaggagacacacacacacagagacagagaggagagagaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagcatagcgctgcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccacgcctgagggcgacatgaacgcgctggccttgagagcaatccggacccacgatcgcttttggcaaaccgaaccggac

SEQ ID NO: 3 - DNA, 477 bp, Unknown
  Rat (Rattus norvegicus) genomic DNA, /promoter region
cagagaggagagagaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagcatagcgctgcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccacgcctgagggcgacatgaacgcgctggccttgagagcaatccggacccacgatcgcttttggcaaaccgaaccggac

SEQ ID NO: 4 - protein, 17 aa, Unknown
  synthetic peptide sequence
GSGSGSGSGSKKKKKKK

SEQ ID NO: 5 - protein, 10 aa, Unknown
  synthetic peptide sequence
CDCRGRDCFC